(19) United States
(12) Patent Application Publication
(10) Pub. No.: US 2003/0022203 A1
(43) Pub. Date: Jan. 30, 2003

(54) Cellular Arrays

(76) Inventor: Rajan Kumar, Robbinsville, NJ (US)

Correspondence Address: Rajan Kumar, 18 Buford Road, Robbinsville, NJ, 08691, US

(21) Appl. No.: 10/131,567

(22) Filed: Apr. 23, 2002

Related U.S. Application Data
(60) Provisional application No. 60/285,566, filed on Apr. 23, 2001.

(51) Int. Cl. C12Q 1/68; G01N 33/53; G01N 33/542; C12M 1/34

(52) U.S. Cl. 435/6; 435/287.2; 435/7.9

(57) ABSTRACT

The invention discloses a novel technique of assaying cultured cells with the use of fluidic devices. The methods and devices disclosed in the invention allow easy introduction and removal of cultured cells and tissues from fluidic devices. The applications of the present invention include cell-based assays for drug discovery, genomics and proteomics.

### CROSS-REFERENCE TO RELATED APPLICATIONS

This application claims the benefit under 35 U.S.C. 119(e) from U.S. Provisional Application 60/285,566, filed Apr. 23, 2001.

### STATEMENT REGARDING FEDERALLY SPONSORED RESEARCH OR DEVELOPMENT

The invention was funded in part by the U.S. Government through a Department of Defense contract (SPAWAR N66001-01-C-8058).

### BACKGROUND OF THE INVENTION

The present invention lies in the field of cellular biology and is particularly concerned with the technique of cell culture in conjunction with the use of fluidic devices for measurement of cell response to analytes in a sample of interest, screening of samples for identifying molecules or organisms that induce a desired effect in cultured cells, and selection and identification of cell populations with novel and desired characteristics.

Cells are fundamental units of life. As such, assays that use live cells as reporters have been used for multiple biochemical applications. Major applications of such as assays include drug discovery, diagnostics, environmental testing and cellular engineering. There is a need to further the development of cell- and tissue-based assay systems by enhancing their longevity, stability, and system integration.

In order to develop the ability to detect very low levels of reagents, the development of cell and multi-cellular tissue-based sensors has been undertaken. Such sensors rapidly detect and predict physiological consequences of both known and unknown reagents by their activities. Due to lack of a requirement for prior knowledge of the threat, such biosensors are particularly suitable for biowarfare defense against unknown and engineered threats. Such devices that respond to a wide range of agents and accurately predict physiological consequences of exposure require multi-cellular assemblies and employ intercellular communications similar to human body's response. The sensitivity and biological signature of cellular response are captured from multiple cell-types including neurons, endothelial cells, immune cells, and stem cells.

Further advances in cell-based assays will result from using recent advancements in engineering functional responses in cells, materials that support the fabrication of multicellular arrays or tissues, and incorporating reporter molecule technologies such as in vivo fluorescence and luminescence. Cells modified to enhance characteristics that make them more suitable for biosensor applications have been demonstrated recently by modifying HEK293 cells to obtain a higher response on field-effect transistors. Innovative methods to pattern cells on substrates have been also reported. The future cell-based assay systems will respond to and report on a wider spectrum of stimuli and provide information on functional responses to agents of interest.

To create tissue-based devices, co-cultures of reporter cells with necessary support cells in three-dimension are required. The major challenge is the ability to culture cells for long duration of times, while maintaining 3-dimensional spatial localization, physiological function and access to nutrients and samples. Prior approaches have included immobilization of tissue slices on arrays of electrodes, and construction of three-dimensional scaffolds. A promising approach to creating a three-dimensional scaffold for neural cell growth is use of hydrogels.

However, a number of major challenges still need to be overcome before functional biosensors based on cell- or tissue-based concepts can be deployed. These include:

Stability of cells and tissues.

Turnover and renewal of functional components.

Nutrient Supply and waste removal.

Collecting signal output from cells and tissues.

Spatial arrangement of cells within the matrix.

Signal processing.

### BRIEF SUMMARY OF THE INVENTION

The invention provides an approach to building microfluidics assay devices by combining cellular arrays with fluidic chips and systems. The arrays of invention are glass or plastic rods/tubes or sheets that have cells grown on them. The arrays are then introduced into microfluidic devices as needed, and can be replaced or reinserted as needed.

### BRIEF DESCRIPTION OF THE SEVERAL VIEWS OF THE DRAWING

FIG. 1 shows an array of the invention 10 being used with a microfluidic device 20.

FIG. 2A shows an array of the invention 10 comprising a single cell-bearing region 30 on its surface.

FIG. 2B shows an array of the invention 10 comprising multiple cell-bearing regions (30A, 30B, . . . , 30H) on its surface.

FIG. 3 shows an assembly 70 of multiple arrays of the invention (10A, 10B, . . . , 10H).

FIG. 4 shows the assembly of multiple arrays of the invention 70 being used in conjunction with a multi-channel fluidic device 80.

FIG. 5A shows another embodiment of the invention 100 comprising a well 110 on a substrate.

FIG. 5B shows the top view of the embodiment 100.

FIG. 5C shows a cross-section of the embodiment 100 through the well 110 and channel 120.

FIG. 5D shows a fluidic chip 150 that can be used with embodiment 100 of the invention.

FIG. 5E shows a fluidic device assembled by putting together embodiment 100 of the invention and fluidic chip 150.

FIG. 6A shows a method of assembling embodiment 100 of the invention and a culture cup 200 for facilitating cell culture in the well of embodiment 100.

FIG. 6B shows a cross-section through the assembled embodiment 100 of the invention and the culture cup.

FIG. 6C shows a cross-section through the assembled embodiment 100 of the invention and the culture cup showing the culture medium 210.

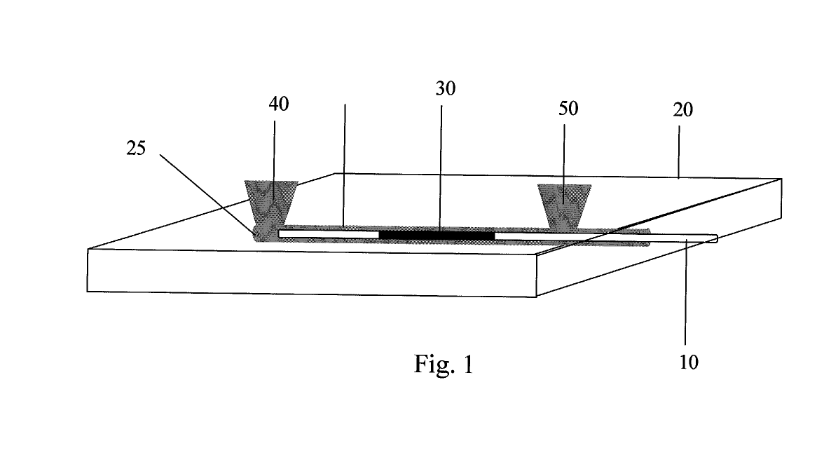

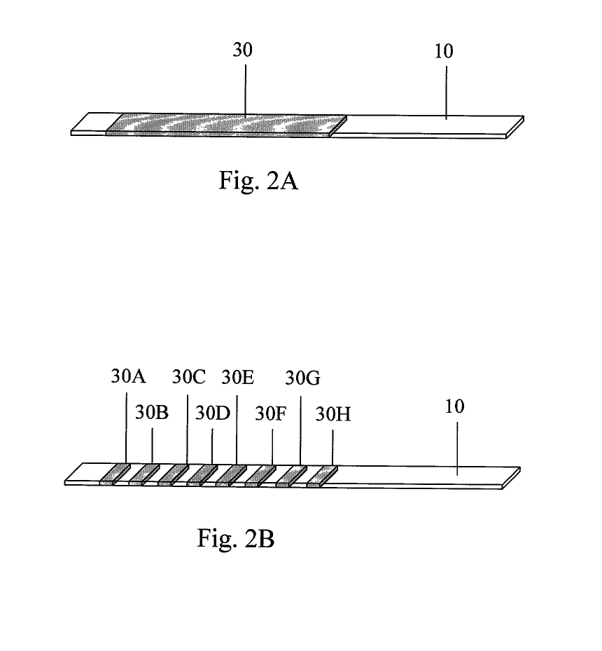

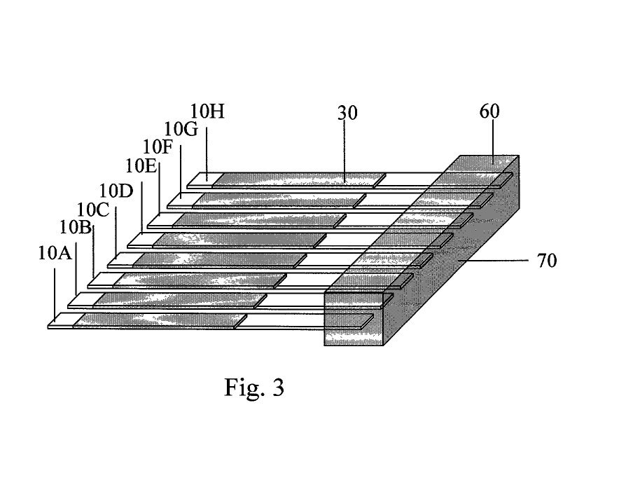

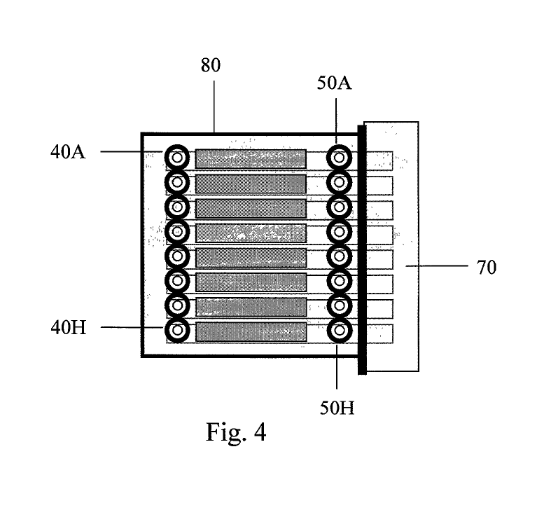

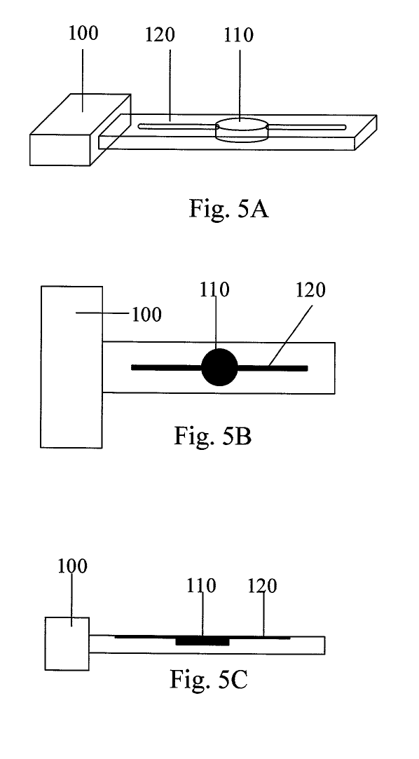

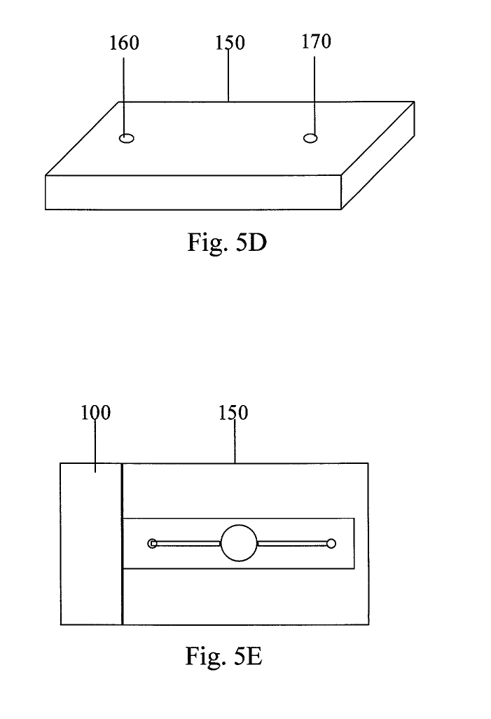

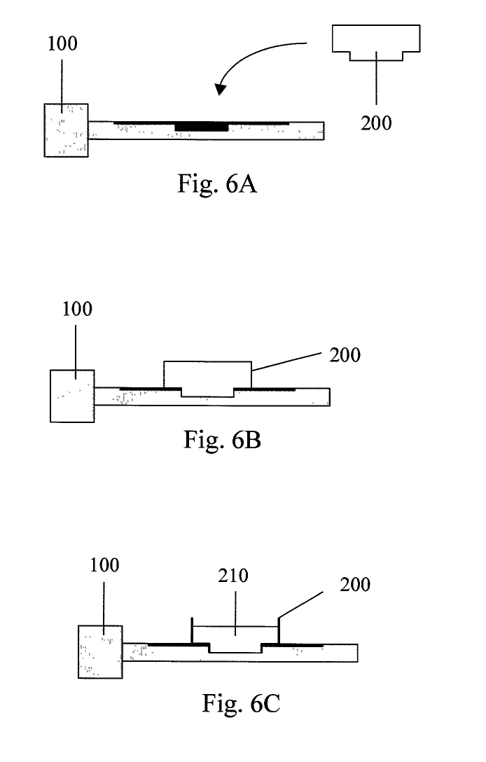

### DETAILED DESCRIPTION OF THE INVENTION

The fundamental basis of the invention is arrays of cells on substrates such as glass or plastic rods/tubes or sheets that are used for customization of re-usable fluidic devices. These arrays can be then assembled into 2D arrays and 3D arrays. The cells grown on the array structures are coupled with re-usable microfluidic systems.

The arrays consist of cells on a suitable substrate. For cellular arrays, rectangular fibers or sheets are used with typical thickness of approximately 100-200 microns. However, rods/tubes and sheets with thickness between 1 micron and 1 mm can be used. The cells will be grown on one or both surfaces of the sheet, and typically one glass substrate will contain one or more types of cells (as a mixed population) grown on it. Additionally, it is possible to generate a discrete array of cells on the substrate by dividing it into multiple circumscribed cell growth areas.

Arrays consisting of multiple substrates can be assembled into 2D arrays that fit into prefabricated fluidics chips. Various 2-D arrays can consist of assemblies of identical arrays or non-identical arrays. The former are designed for fluidics chips that perform analysis of multiple samples against a similar set of cells; the latter are designed for analysis of a large number of cell types or probes against one or more samples.

2-D arrays can be assembled into 3-D arrays. 3-D cell arrays are ideal for design of tissue-based biosensors, because a number of different cell-types can be included. Additionally, the substrates (rods or sheets) can be used to retrieve the cell response from the three-dimensional collection of cells; for example, by fluorescence light piping from the cells. The 3-D approach can also be used for analysis of multiple samples against multiple non-identical arrays.

The process for using all of these arrays is as follows:

The arrays are introduced into generic chips to generate customized chips and sealed inside. The arrays are either completely sealed inside the device or are protruding out. After array introduction, the chip is used for performing assays. For detection, the array is removed from the chip and the fluorescence of the targets captured on the array is measured. Alternatively, the detection can be performed with the array still enclosed in the chip. After removal of the array, the chip is prepared for re-use by cleaning, and introduction and sealing of a new array. The arrays that protrude from the side of the chip are preferable due to ease of their introduction and removal from the chips.

The cell arrays will be created on Pyrex Borosilicate glass sheets. However, other substrates including various plastics can be used. The glass sheets in thickness as little as 30 micron are commercially available. However, glass sheets with thickness of 100-250 micron will typically be used. To avoid handling of individual glass sheets, virtually all processing will be done with the arrays assembled into 2D arrays. A large number of arrays are laid parallel to each other and immobilized in a mold to generate a 2-D array.

The 2-D cell array are fabricated as follows. The surface of the arrays is coated with poly-L-lysine or other cell adhesion promoting molecules where the cells are required to grow. The assembled array is placed in a cell culture dish. After an appropriate incubation period, cells have grown on the desired areas. Now the arrays can be used as needed. Other methods of creating the arrays can be envisioned and include capture of cells from cell suspensions by using e.g. antibodies.

The 3D arrays will be assembled from multiple 2D arrays. The major challenge in creating 3D arrays is how to spatially localize the cells with in the scaffold. One level of spatial localization will be achieved by distributing the coated and uncoated surfaces on the arrays. Additionally, molecules and coatings that selectively capture particular cell types can be used. An intriguing possibility (and an advantage of the proposed approach) is to introduce 3-D array into a multi-channel 3-D fluidic device so that specific arrays come in contact with specific cells.

The methods to make and use fluidic chips have been described and are widely known. Typically, microfluidic chips for 2-D and 3-D cell arrays are fabricated from plastics. The fabrication process involves joining of two plates, 1) a bottom plate which has etched channels and reaction wells, and 2) a top plate which has drilled holes to provide access to the channels in the bottom plate. For introduction of the arrays, the channels can be accessed from the side of the chip. The channels are etched in plastic substrates by machining or by using molds. The holes in the top plate are generally drilled mechanically. The channel dimensions for typical cell arrays will be 5-10 mm micron wide and 500 microns to 1000 microns deep. Other dimensions can however be envisioned.

The functional response of cells to agents can vary from discrete, morphological changes such as proliferation (cell division and multiplication), and cell death (apoptosis), to subtle changes such as change in gene or protein expression. Given the utility of cell-based assays to provide information with physiological insight, a number of assays to measure all of these responses have been devised and tested. In addition, biosensors have relied on measurement of physical parameters of the cells in response to analytes, such as temperature, metabolic rate, impedance, intracellular and extracellular potentials. All of these methods can be used with the cell arrays of the invention. Two exemplary molecular reporter systems are identification of cells undergoing apoptosis and measurement of intracellular calcium.

One of the common responses on exposure to toxins is cell death or apoptosis. The features of apoptosis include early changes in response to toxins, as well as more delayed and irreversible changes. Since different cell-types might undergo apoptosis in response to different toxins, a monitoring of the apoptotic response of multiple cell types can provide a comprehensive toxic profile of a sample.

Cells exposed to apoptotic stimuli release cytochrome c from mitochondria into the cytosol and initiate a protease cascade. In the cytosol, cytochrome c interacts with apoptotic protease activating factor-1 (Apaf-1). The cytochrome c/Apaf-1 complex cleaves the inactive caspase-9 proenzyme to generate the active enzyme. Activated caspase-9 then initiates the proteolytic activities of other downstream caspases, including caspase-3 and caspase-6. These caspases degrade a variety of substrates, resulting in the systematic disintegration of the cell and results in apoptosis.

Assaying caspase activity in vitro and in vivo by induction of fluorescence provides a simple way to detect cells undergoing apoptosis. In vitro caspase assays use activated caspases for the cleavage of a specific fluorescent substrate and measure change in fluorescence. Fluorometric assays for caspase 3, 8, and 9 are commercially available. For in vivo detection of caspase activity, cell permeable fluorogenic caspase substrates are used. Alternatively, fluorescence resonance energy transfer between green fluorescent protein variants is used to measure intracellular caspase activity.

Intracellular calcium is an important messenger molecule and therefore, an excellent indicator of cellular health. Additionally, intracellular calcium dependent pathways are potentially involved in mediating the action of toxins. Intracellular calcium is assayed using cell membrane permeable dyes including fluorescent dyes e.g. Fura-2. Certain ionophores can be used to increase the intracellular calcium concentration. We plan to use ionophore A23187 and Fura-2 dye to measure cell response based on intracellular calcium concentrations.

Primary substrate used will be Pyrex borosilicate glass. Other materials, both glass and plastics can be used as potential array substrates. Selection criteria will include the ease of handling, efficiency of cell growth, and interference with assay such as background fluorescence emission. The arrays will be coated with poly-L-lysine for attachment of cells to substrates. The arrays can be fabricated with different number of cells and different types of cells to provide an optimal cell culture conditions.

A fluidic device for testing arrays will be fabricated. The size of the chips, the length and dimensions of channels, channel spacing, and thickness of substrates used will be defined for optimum compatibility with arrays. Fluid inlet and outlet tubes will also be inserted in to inlet and outlet ports of the chip. Temperature regulation of the chip is planned by placing it in a cell culture chamber, thus design of the fluidic device will take compatibility with this chamber in to account as well. The assembly of Assay Test System will bring together a number of components developed in earlier tasks, including arrays and sealing method, and will integrate experimental observations including the features of fluidic devices, lengths and dimensions of channels, and pressure required for sealing.

A method to create a watertight seal between the array and the fluidic device will be demonstrated and optimized. Pros and cons of attaching the seals to the chips vs. arrays will be investigated. Arrays containing O-rings will be fabricated and fluidic chip designs for tight sealing and easy introduction of arrays will be tested. Amount of pressure required to create the seal and number of times an O-ring can be used to seal arrays will be determined. The introduction of the sealing method into the assembly process is also a consideration. During this task, we will investigate the high-density printed circuit board and multi-chip module connectors to optimize the reliable attach and release methods between the fluidic chip and the GeneCard array.

### EXAMPLE 1

### A Prototype Cell Array

The array will consist of an assembly of 8 individual borosilicate glass sheets, approximately 5 mm wide and 50 mm long. An area of 5 mm by 25 mm on each sheet will be marked with hydrophobic pen and used for culture of human fibroblast cell line—NIH-3T3. After culture of the cells, the array will be introduced into an eight channel fluidic chip. The cell array will interface with a 75 mm square fluidic chip. The cell arrays will be evaluated for the quality of cells cultured on arrays. The number of cells per unit area, the viability of cells, the response to growth factors, and the number of floaters will be measured as indicators of the quality of the cells.

### EXAMPLE 2

### A Prototype Array-chip Interface

The array chip interface will consist of a water-tight seal between the chip material, and the material used to assemble the array. The seal will consist of a gasket or individual O-rings. Nutrients will be supplied to the cells by a syringe pump fluidic station. Same station will be used to introduce test sample to the cells. Evaluation will include the number of times a chip can be re-used to interface with arrays, control of environmental parameters to maintain cell viability and health, measure the volume of culture chamber and volume of replacement medium, the thickness of the sheet used for cell culture, incorporation of signal transduction methods into the interface, and separation of signal captured from different areas of the array.

### EXAMPLE 3

### Capture of Cell Response

The primary approach for measuring cell response will be optical imaging of cultured cells for fluorescence. Fura-2 dye will be used to measure fluorescence based on intracellular calcium. An Ca(2+)-ionophore A23187 will be used to change the intracellular concentration of calcium. Another example will be to capture fluorescence signal from a reporter system that produces fluorescence in response to induction of apoptosis. Other methods of capture of signal—electric response with electrodes imprinted on the array elements, thermal measurements, and using array elements as optical conduits will be evaluated.

### EXAMPLE 4

### Microfluidic Assay

The arrays will be tested in the microfluidic chips. Apoptosis with on-chip analysis will be performed using mammalian cells. The beginning of apoptosis will be analyzed by a fluorescence assay, based on fluorescence resonance energy transfer and detects the activity of intracellular caspases. 293 cells will be obtained from ATCC. Cells will transfected with FRET constructs sensitive to caspase activity. Apoptosis will be induced in 293 cells using tumor necrosis factor-alpha. The fluorescence level of the cells will be recorded. The concentrations of TNF-alpha and time it takes for apoptosis will be analyzed. Control experiments will be done in parallel with the exposure to TNF-alpha.

Apoptosis with off-chip analysis will be performed for activation of caspase cascade and for the presence of characteristic ladder resulting from cleavage of chromosomal DNA. The cells induced to undergo apoptosis as in example 1 will be withdrawn from the device by removing the substrate carrying the cells. The cells will then be lysed. The cell lysates will be tested for the activity of caspase 3, 8 and 9 using ApoAlert kits from Clontech. The presence of a DNA ladder will be detected by end-labeling of fragmented DNA with a technique called TUNEL using a commercially available kit. Correlation between off-chip and on-chip analyses will be performed. Also the sensitivity and specificity of the two approaches will be compared.

Intracellular calcium assay will be performed on-chip by measuring fluorescence from Fura-2 loaded into NIH 3T3 cells. The intracellular calcium concentration will be modified by exposing cells to A23187.

### EXAMPLE 5

### 3D Arrays

An 8×8 scaffold will be fabricated. Glass sheets will be used similar to those for GeneCard array. The dimensions of the scaffold will be approximately 75 mm long, 25 mm wide and 6 mm thick. The assembly of 3D scaffold from 2D GeneCard will be demonstrated. Deposition of adhesion molecules or coatings to specified locations will be performed. The ability to customize each element of the scaffold after the scaffold has been assembled by introducing specific array element into a microfluidic device will be demonstrated. The resulting scaffold will be populated with cells and used to assay samples. Fluorescent assays for intracellular calcium and for induction of apoptosis will be performed, as described for 2-D arrays. The ability to resolve fluorescent cells from non-fluorescent cells in three-dimensional arrangement of cells will be evaluated. Alternatively, using the array elements themselves to capture and transmit the signal by wave-guiding will be used. Attempts to form defined tissue structures by using different combination of cells will be made.

What I claim as my invention is

1. Any fluidic device comprising
a fluidic chip, and
a substrate containing a pre-defined cellular array inserted into the fluidic chip.

2. Any substrate containing a pre-defined cellular array used with a device of claim 1.

3. Any fluidic device of claim 1 comprising insertion of a substrate containing a pre-defined cellular array in to a fluidic chip for further processing or analysis.

4. Any fluidic device of claim 1 comprising removal of a substrate containing a pre-defined cellular array from a fluidic chip for further processing or analysis.

5. Any analysis method comprising detection of cell characteristics after removal of a substrate containing a pre-defined cellular array from a fluidic chip.

6. Any analysis method comprising removal of an cellular array of claim 2 from a fluidic chip and insertion of one or more other similar or different arrays in to the same chip for further processing or analysis.

7. An array of claim 2 comprising the substrate is rod shaped, sheet-shaped or a 3D solid shaped.

8. An array of claim 2 comprising the cells are distributed on one, both or all sides of a substrate.

9. An array of claim 2 comprising of more than one substrate such that the array members are distributed in two or three space dimensions.
